(19) United States
(12) Patent Application Publication
(10) Pub. No.: US 2020/0147347 A1
(43) Pub. Date: May 14, 2020

(54) VASCULAR RE-ENTRY CATHETER

(71) Applicant: ORBUSNEICH MEDICAL PTE. LTD., SINGAPORE (SG)

(72) Inventor: Robert J. COTTONE, Davie, FL (US)

(21) Appl. No.: 16/712,333

(22) Filed: Dec. 12, 2019

Related U.S. Application Data
(63) Continuation-in-part of application No. 16/594,941, filed on Oct. 7, 2019.
Application No. 16/594,941 is a continuation-in-part of application No. 15/726,024, filed on Oct. 5, 2017.
Application No. 16/594,941 is a continuation of application No. 14/854,242, filed on Sep. 15, 2015, now abandoned.

(51) Int. Cl. A61M 25/01; A61M 25/00

(52) U.S. Cl. CPC A61M 25/0194; A61B 2017/22095; A61M 2025/0197; A61M 25/0068

(57) ABSTRACT

A catheter device having a distal tube portion having a longitudinal axis and a tube wall comprising at least one side port; and a guide tip mounted on the distal tube portion, the guide tip defining: a unitary body having an outer wall, and a plurality of indentations in the outer wall, wherein the indentations are oriented substantially parallel to the longitudinal axis, and wherein each indentation extends from a distal-most end of the guide tip to a proximal region of the guide tip.

### CROSS-REFERENCE TO RELATED APPLICATION

This application is a continuation-in-part of U.S. patent application Ser. No. 16/594,941, filed Oct. 7, 2019, which is a continuation-in-part of U.S. patent application Ser. No. 14/854,242 ('242 application), filed Sep. 15, 2015, entitled “Vascular Re-entry Catheter.” The applications, in turn, claim priority to U.S. provisional application No. 62/050,456, filed Sep. 15, 2014, and to U.S. provisional application No. 62/060,152, filed Oct. 6, 2014. This application also claims priority to U.S. patent application Ser. No. 15/726,024, filed Oct. 5, 2017, entitled “Modular Vascular Catheter.” The disclosure of all of the preceding applications is hereby incorporated by reference in their entirety.

### STATEMENT REGARDING FEDERALLY SPONSORED RESEARCH OR DEVELOPMENT

n/a

### BACKGROUND OF THE INVENTION

Chronic Total Occlusion (“CTO”) is a complete or near complete blockage of a blood vessel, such as a coronary artery. As many as 30% of the patients with coronary artery disease have CTOs somewhere through the left or right arterial system. Traditionally CTO has usually been treated by a bypass procedure where an autologous or synthetic blood vessel is anastomotically attached to locations on the blood vessel upstream and downstream of the occlusion. While effective, such bypass procedures are quite traumatic to the patient.

Recently, catheter-based intravascular procedures have been developed to treat CTO with improved success rates. Such procedures include angioplasty, atherectomy, stenting, and the like, and the catheters are usually introduced percutaneously. Treatment of CTO percutaneously significantly reduces the need for surgery (coronary artery bypass graft—CABG). Moreover, CTO percutaneous coronary intervention (PCI) can result in symptomatic relief for the patient, re-establishing coronary blood flow, improved left ventricular function and potentially, survival advantage. Peripheral vascular occlusions outside of the coronary vascular anatomy are also treatable with such interventions.

Before such catheter-based treatments can be performed, it is usually necessary to cross the occlusion with a guidewire to provide access for the interventional catheter. Available techniques for crossing the occlusion generally fall into two approaches: the antegrade approach, which involves crossing a wire from the proximal end to the distal end of the occlusion (either through the CTO directly or through the subintimal space), and the retrograde approach, which refers to CTO access of the distal cap via collateral vessels. The latter is usually reserved as a second line strategy for failed intimal antegrade crossing.

For treating CTO by PCI with antegrade access, a number of different devices have been developed, including the CrossBoss™ and Stingray™ systems. See http://www.bostonscientific.com/en-US/medical-specialties/interventional-cardiology/procedures-and-treatments/coronary-chronic-total-occlusion-system.html (last accessed Sep. 10, 2015); see also, U.S. Pat. Nos. 8,632,556, 8,202,246, 8,636,712, 8,721,675, 6,511,458.

The CrossBoss™ catheter can be first used to facilitate the crossing a CTO as simply bluntly dissecting via small micro channels within the vessel through the occlusion or if this is not successful in crossing, the device can navigate from the subintimal space of a vascular wall. As an illustration, FIG. 1A shows a schematic representation of a CrossBoss™ catheter 100, which includes a rounded/blunt distal tip 108 mounted to a flexible proximal shaft 120 which is torquable through rotation of the handle 130, the shaft 120 having a lumen which accommodates a guidewire 102.

The Stingray™ catheter, Stingray™ Guidewire can be used subsequent to the CrossBoss™ catheter to facilitate orienting and steering a guidewire or reentry device from the subintimal space into the true lumen of an artery. As an illustration, FIG. 1B shows a schematic representation of a Stingray™ catheter having a distally positioned laterally inflatable balloon 210 and a proximal shaft 220 having a central guidewire lumen 225. The side-ports 212 and 214 are located on opposite sides of a portion of the central lumen flanked by the balloon 210 and identified by radiopaque markers 232 and 234. The side ports 212 and 214 communicate with the central guidewire lumen 225 and facilitate the steering of the reentry device 240 with a pre-biased tip (at an angle to the central lumen) by allowing the tip of the Stingray™ Guidewire reentry device 240 to exit from the catheter from one of the side ports.

The CrossBoss™ catheter can be used to pass through the proximal cap of a CTO by rotation of the blunt tip. However, if this is not successful, a procedure employing both the CrossBoss™ and Stingray™ catheters to cross the CTO would be needed. The procedure can be generally described as follows.
(1) advancing the CrossBoss™ catheter over a guidewire to an interface between the catheter distal tip and surrounding tissue;
(2) penetrating the catheter tip into the vascular wall and advancing the CrossBoss™ device within the vascular wall to establish a channel in the subintimal space of the vascular wall such that the tip extends longitudinally across the occlusion;
(3) withdrawing the CrossBoss™ catheter over the guidewire;
(4) advancing the Stingray™ catheter over the guidewire;
(5) inflating the balloon of the Stingray™ catheter to cause the Stingray™ balloon to assume one of two orientations;
(6) withdrawing the guidewire from the Stingray™ catheter;
(7) advancing a reentry device having a pre-configured tip portion in a compressed state into the lumen of the Stingray™ catheter; and
(8) manipulating the tip of the reentry device with the aid of radiographic visualization such that the tip of the reentry device exits from one side port of the Stingray™ catheter in a natural state and into the lumen of the artery (true lumen).

The Stingray™ catheter can then be withdrawn, leaving the reentry device in place which establishes a pathway from the proximal segment of the vascular lumen and the distal segment of the vascular lumen, over which a balloon catheter can be subsequently introduced and deployed at the site of the CTO. A stent can be further implanted at the site which has been expanded by the balloon.

While the above procedure of subintimal crossing using a combination of CrossBoss™ and Stingray™ catheters overcame certain difficulties of previous generation techniques in direct antegrade CTO access, the procedure is complex and time-consuming. Furthermore, switching from CrossBoss™ to Stingray™ can introduce unintended errors. For example, withdrawing the CrossBoss™ catheter from the guidewire (so that the Stingray™ catheter may be introduced over the guidewire) may cause the guidewire to shift position in the subintimal space, or worse, to retract from the subintimal space, in which case the operator would not be able to properly introduce the Stingray™ catheter over the guidewire into the subintimal space. As a result, the previous step of using CrossBoss™ catheter to cross the subintimal space would need to be repeated. Additionally, advancing and inflating the distal balloon of the Stingray™ catheter in the subintimal space can create excessive delamination and substantial trauma in the layers of the vascular wall.

Another issue associated with such subintimal exploration is the difficulty of determining the precise orientation of the catheter as it moves through the tortuous anatomy of the coronary artery and its sub-branches. A mistaken reading of the reentry catheter orientation can lead to an iatrogenic puncture of the pericardium, or other complications when the reentry catheter is guided in the wrong direction with respect to the artery lumen.

It would be desirable to provide devices and methods for treatment of vascular conditions associated by crossing a CTO in a blood vessel by exploring the subintimal space in a more simplified procedure with reduced error rate and reduced trauma to the blood vessel, and to provide devices and methods that promote the safety of CTO procedures by aiding in the ready and accurate determination of reentry catheter orientation with respect to the vasculature and cardiac tissue.

### SUMMARY OF THE INVENTION

In one aspect, the present invention provides a catheter device. The catheter device includes a distal catheter tube portion having a longitudinal axis and including a tube wall comprising at least one side port, at least one radiopaque marker, and at least one wing protruding radially outward from the tube wall.

In some embodiments, the tube wall includes two wings protruding radially outward in diametrically opposing directions. In certain of these embodiments, wherein the at least one side port is radially offset from each of the two wings for about 90 degrees. In specific embodiments, the first side port and the second side port are radially offset for about 180° degrees from each other.

In some embodiments, the at least one side port is beveled.

In some embodiments, the catheter device includes a radiopaque marker affixed on the distal catheter tube portion in axial alignment with the at least one side port. In other embodiments, the catheter device includes a radiopaque marker encircling the at least one side port.

In some embodiments, the tube wall includes a first side port and a second side port which is longitudinally and radially offset from the first side port, the second side port being located distal the first side port. In certain of these embodiments, the tube wall comprises a first radiopaque marker located longitudinally between the first side port and the second side port, and a second radiopaque marker distal the second side port.

In some embodiments, the at least one wing is part of a guide-tip engaging a distal end of the catheter. In other embodiments, the at least one wing can be placed in a distance from the distal end of the catheter.

In some embodiments, the catheter device includes at least one spiral-cut section, and the at least one side port is located in the spiral-cut section.

In certain embodiments, the catheter device includes at least two spiral-cut sections having different pitches.

In some embodiments, the catheter device includes at least one spiral-cut section with interrupted spirals.

In certain embodiments, the catheter device includes at least two interrupted spiral-cut sections having different pitches.

The at least one wing can be formed from a polymeric material, a metal, or a composite material.

In another aspect, the present invention provides a method for facilitating treatment of an occlusion in a blood vessel with a catheter device as described herein. The blood vessel has a vascular wall defining a vascular lumen containing an occlusion therein. The occlusion separates the vascular lumen into a proximal segment and a distal segment. The catheter device has a lumen and includes a distal catheter tube portion including a tube wall comprising at least one side port and at least one radiopaque marker, and a guide-tip located at a distal end of the catheter, where the guide-tip includes at least two wings protruding radially outward in diametrically opposing directions. The method includes: positioning the catheter device proximate the occlusion; advancing the guide-tip within the vascular wall adjacent the occlusion until the at least one side port is positioned distal of the occlusion to establish a channel in the vascular wall extending longitudinally across the occlusion; orienting the at least one side port toward the vascular lumen; inserting a re-entry device through the lumen of the catheter device wherein the re-entry device has a distal end portion in a compressed state; and manipulating the re-entry device such that a distal end portion of the re-entry device exits, in a natural state, from the at least one side port into the distal segment of the vascular lumen.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings wherein:

FIG. 1A schematically depicts a CrossBoss™ catheter which is known in the art.

FIG. 1B schematically depicts a Stingray™ catheter which is known in the art.

FIG. 2A shows a catheter (tube) having a guide-tip with wings, and a side port in a spiral-cut section according to one embodiment of the present invention.

FIG. 2B shows a front view of the guide-tip of the catheter shown in FIG. 2A.

FIG. 2C is a side cross section view of a portion of the catheter shown in FIG. 2A.

FIG. 2D is a front view of a guide-tip with wings according to one embodiment of the present invention.

FIG. 2E is a front view of a guide-tip with wings according to another embodiment of the present invention.

FIG. 2F is a side cross section view of a portion of a catheter having a winged guide-tip according to another embodiment of the present invention.

FIG. 2G is a side cross section view of a portion of a catheter having distal wings according to another embodiment of the present invention.

FIG. 3A is a photo of a guide-tip as illustrated in FIGS. 2A and 2B.

FIG. 3B is a back view of the guide-tip as illustrated in FIGS. 2A and 2B.

FIGS. 3C and 3D are cross section views of the guide-tip along lines A-A and B-B indicated in FIG. 3A.

FIG. 3E is a cross section view of a guide-tip according to another embodiment of the present invention.

FIG. 3F shows exemplary side cross section wing shapes according to some embodiments of the present invention.

FIG. 3G is a front view of a guide-tip having more than two wings according to some embodiments of the present invention.

FIG. 3H is a front view of a guide-tip having an anisotropic transverse cross section shape according to some embodiments of the present invention.

FIG. 3I is a front perspective view of a guide-tip according to some embodiments of the present invention.

FIG. 3J is a front perspective view of another guide-tip according to some embodiments of the present invention.

FIG. 3K is a front perspective view of still another guide-tip according to some embodiments of the present invention.

FIGS. 4A and 4B are schematic side views of a catheter having radiopaque markers according to some embodiments of the present invention.

FIGS. 4C-4R provide additional views of catheters and components thereof having radiopaque markers according to some embodiments of the present invention

FIG. 5A is a top view of a beveled port on a spiral-cut section of a catheter according to one embodiment of the present invention.

FIG. 5B is a side cross sectional view of the beveled port as shown in FIG. 5A.

FIG. 6 shows a catheter containing a plurality of spiral-cut sections on a distal catheter tube portion according to some embodiments of the present invention.

FIG. 7A is a side view of spiral-cut section of a catheter including interrupted spirals according to an embodiment of the present invention.

FIG. 7B depicts a section of a catheter having an interrupted spiral-cut pattern in an unrolled condition according to one embodiment of the present invention.

FIGS. 8A-8D are photographs of different spiral-cut portions of a catheter according to an embodiment of the present invention.

FIG. 9A is an exploded view of components of a handle assembly for use with a catheter according to some embodiments of the present invention.

FIG. 9B depicts the handle assembly as assembled in a first configuration from the components shown in FIG. 9A.

FIG. 9C depicts the handle assembly as assembled in a second configuration from the components shown in FIG. 9A.

FIG. 10A depicts a configuration of a proximal portion of a catheter according to some embodiments of the present invention.

FIG. 10B is a front view of a component of the handle assembly shown in FIG. 9A.

FIG. 10C depicts various cross section configurations of a proximal portion of a catheter according to some embodiments of the present invention.

FIG. 11 depicts a configuration of a catheter after passing a CTO lesion from the subintimal space and a re-entry device reentering the vascular lumen from a side port of the catheter according to some embodiments of the present invention.

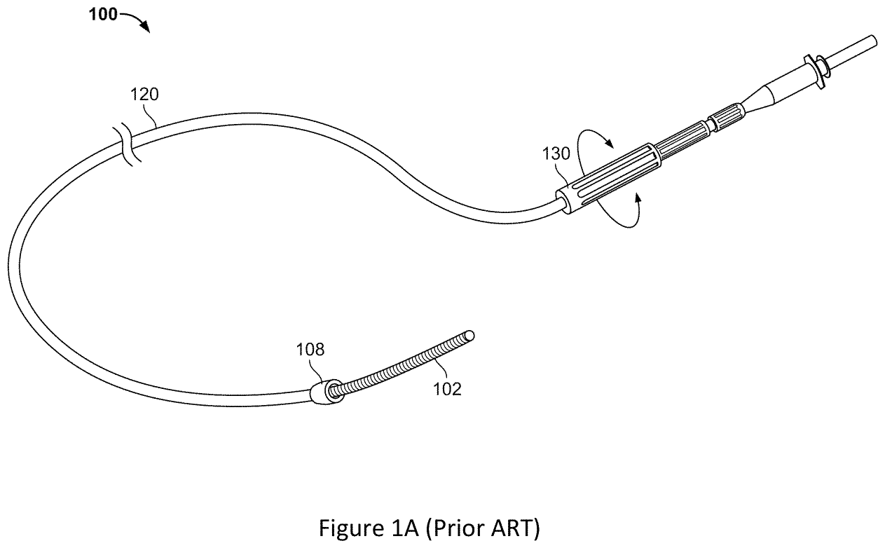

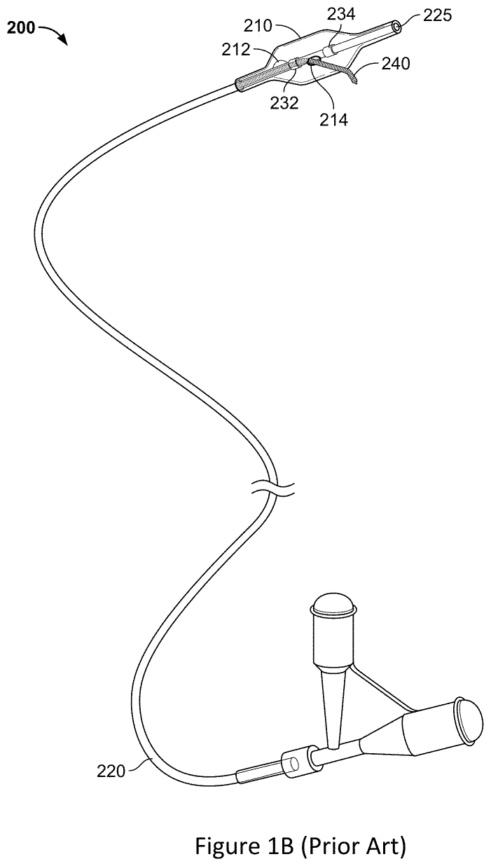

### DETAILED DESCRIPTION OF THE INVENTION

In one aspect, the present invention provides a catheter device (or catheter). The catheter can be used for the treatment of CTO, either by passing the CTO directly, or by passing the CTO via the subintimal space. In another aspect, the present invention provides a method for treating CTO.

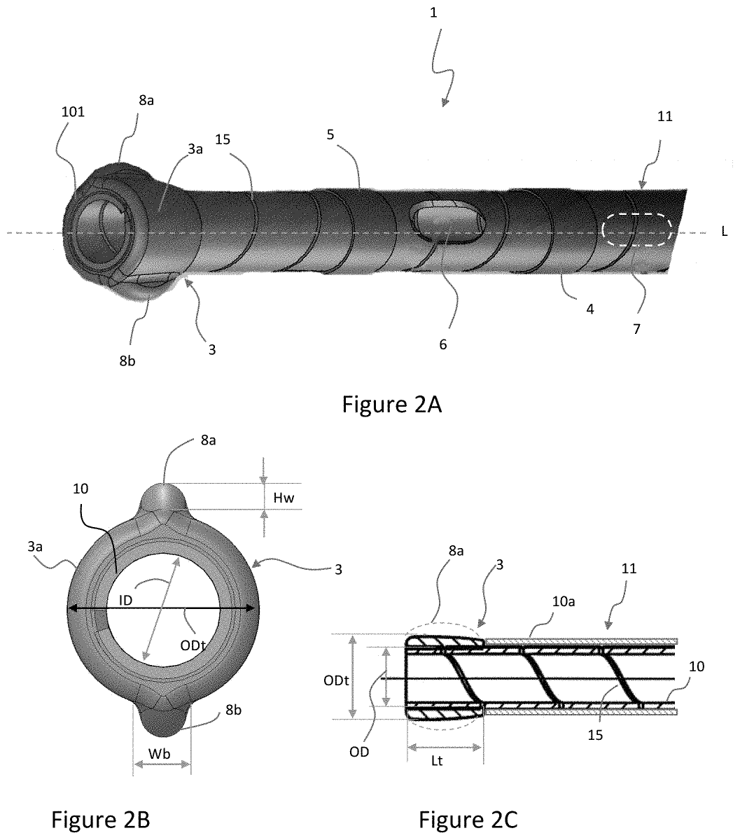

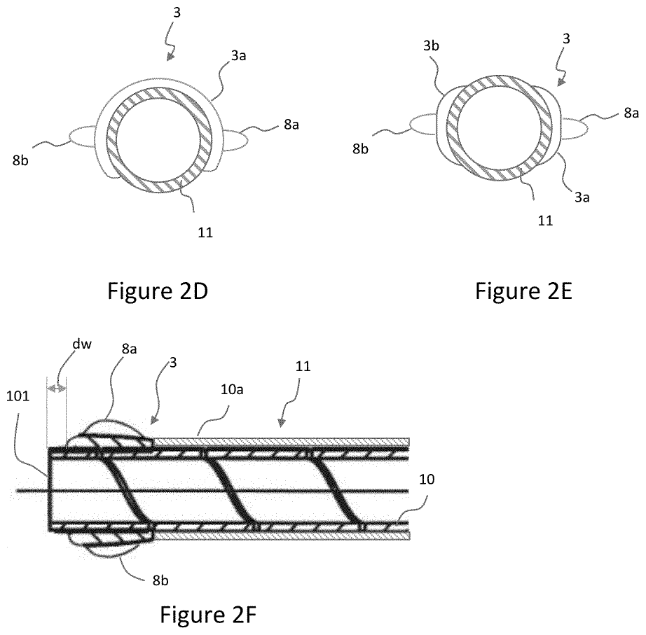

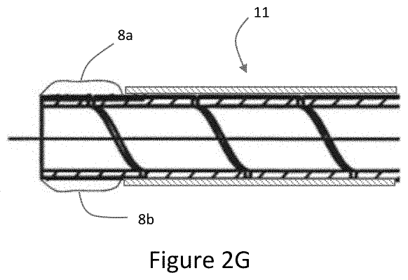

As illustrated in FIGS. 2A and 2B, a catheter device 1 according to one embodiment of the present invention includes a distal tube portion 11 having a tube wall 10 and a longitudinal axis L. The tube wall 10 is cylindrical and has an inner surface that defines an inner lumen, and an externally-facing outer surface. The outer surface has circular circumference defining a plane that is orthogonal to the longitudinal axis. Any point on the outer surface of the tube wall 10 can be defined by a combination of longitudinal and circumferential position.

On the distal end 101 of the catheter 1 (which is also the distal end of the distal tube portion 11) is a blunt guide-tip (or tip) 3, which encircles a distal end portion of the distal tube portion 11. Guide-tip 3 includes a base portion 3a, as well as two lateral wings 8a and 8b radially protruding outward from the circumference of tip 3. In certain embodiments, the guide-tip 3 wing can include fewer or more wings, e.g., only one wing, or greater than two wings, as described herein.

When two wings are present, they can be radially separated or offset by an angle of about 30 to about 90 degrees, or from about 90 to about 180 degrees or any fraction in-between. For example, the angle can be about 30 degrees, about 60 degrees, about 90 degrees, about 120 degrees, about 150 degrees or about 180 degrees. As shown in FIGS. 2A and 2B, in some embodiments, the two wings 8a and 8b can be positioned approximately diametrically opposed around the guide-tip, i.e., about 180 degrees from each other ±10 degrees, more preferably ±5 degrees.

FIG. 2C shows a cross section view of a portion of the tube 1 of FIG. 2A along the longitudinal axis L. As shown in FIGS. 2B and 2C, the outer diameter of the guide-tip ODt (which does not include the wings) is greater than the outer diameter (OD) of the distal tube portion 11, which is greater than the inner diameter (ID) of the distal tube portion 11. The wings have a base width Wb, a height Hw measured from OD to the peak of the wings. The leading edges of the wings are generally rounded or smooth.

As shown in FIG. 2B, the guide-tip 3 can, in certain embodiments, completely encircle a circumference of distal tube portion 11. In alternative embodiments, and as illustrated in FIGS. 2D and 2E, the guide-tip 3 (including base 3a and wings 8a/8b) does not completely encircle of the distal tube portion 11. For example, the guide-tip 3 only encircles three quarters, two thirds, one quarter or smaller percentages of the circumference of the distal tube portion 11. In certain embodiments, and as illustrated in FIG. 2E, the guide-tip 3 may include multiple separate base parts (3a, 3b) distributed along the circumference of the distal tube portion 11.

In certain embodiments, the guide-tip 3 with wings 8a and 8b may be positioned slightly away from the distal end 101 of the tube portion 11, e.g., at a distance dw ranging from about 1 to about 100 mm (see FIG. 2F), e.g., about 10 mm to about 75 mm, or about 25 to about 50 mm.

In certain embodiments, one or more wings can be directly joined to the tube without being supported by a base portion of a tip, e.g., at the distal end 101, and/or away from the distal end 101. As illustrated in FIG. 2G, wings 8a/8b are directly joined to the tip of the distal tube portion 11 (e.g., by welding, adhering, etc.) without being a part of a tip encircling a distal tube portion 11. In such situations, the wing(s) itself can also be considered as the only component of a guide-tip.

Depending on the material as well as the structural requirements in terms of flexibility, the thickness of the tube wall 10 can vary, e.g., from about 0.002 inch to about 0.02 inch, or from about 0.05 mm to 2 mm, e.g., 0.05 mm to about 1 mm, about 0.1 mm, 0.2 mm, 0.3 mm, 0.4 mm, 0.5 mm, 0.6 mm, 0.7 mm, 0.8 mm, 0.9 mm, 1.0 mm, etc. The inner diameter of the lumen (ID) of the distal tube portion 11 can vary, e.g., from about 0.01 inch to about 0.04 inch, or from about 0.1 mm to about 2 mm, or from about 0.25 mm to about 1 mm, e.g., about 0.2 mm, about 0.3 mm, about 0.4 mm, about 0.5 mm, about 0.6 mm, about 0.7 mm, about 0.8 mm, about 0.9 mm, about 1 mm, etc. The outer diameter of the lumen (OD) of the distal tube portion can also vary, e.g., from about 0.2 mm to about 3 mm, e.g., about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, etc. The thickness of the tube wall, the inner diameter ID and the outer diameter OD can each be constant throughout the length of the catheter, or vary along the length of the catheter.

In certain embodiments, the height of the wings Hw can range from about 5%-about 50% (including about 10% to about 40%, about 15% to about 30%, or about 20%) of the outer diameter ODt of the tip 3; alternatively, the height of the wings Hw can be about 10% to about 15%, about 15% to about 30%, or about 5% to about 45% of ODt. In some embodiments, the base width of the wings Wb can be about 5%-30% of the outer diameter ODt of the tip 3. The axial length of the base of the wings can be approximately the same as the axial length Lt of the guide-tip 3 (see FIG. 2C), and can range from about 5 mm to about 20 mm, about 7.5 mm to about 15 mm, about 8 mm to 12 mm, or about 10 mm. Alternatively, the axial length of the base of the wings can be smaller than the axial length Lt of the guide-tip 3.

The tube wall 10 and guide-tip 3 may be formed from a metal, a polymer or a composite material. Suitable metals can include cobalt-chromium, stainless steel, MP35N, nickel titanium, etc., as well as metal alloy such as a shape memory material, e.g., Nitinol. Alternatively, the tube wall may be composed of polymers such as aliphatic polyether-urethanes, polyamides, low-density polyethylene (LDPE), polypropylene or mixtures of polymers. The tube wall distal tube portion may also be formed from a composite of polymers and metal, such as a composition of joined or abutted metal and polymer forming a generally tube like structure. The distal catheter tube portion can be formed from metal, e.g., stainless steel. The guide-tip 3 and/or the wings 8a/8b can be made of the same material as the tube wall or different materials. For example, the guide-tip may include a radiopaque material such as a radiopaque filler composition. The guide-tip may include metal and a softer outer component such as a polymer varying in udometer from soft rubber like materials to hard composite polymer or plastics. Also, the wings may be made from the same or different material as the remainder of the guide-tip. For example, the wings may be formed from a polymeric material, a shape memory material such as Nitinol, or a metal such as cobalt chromium.

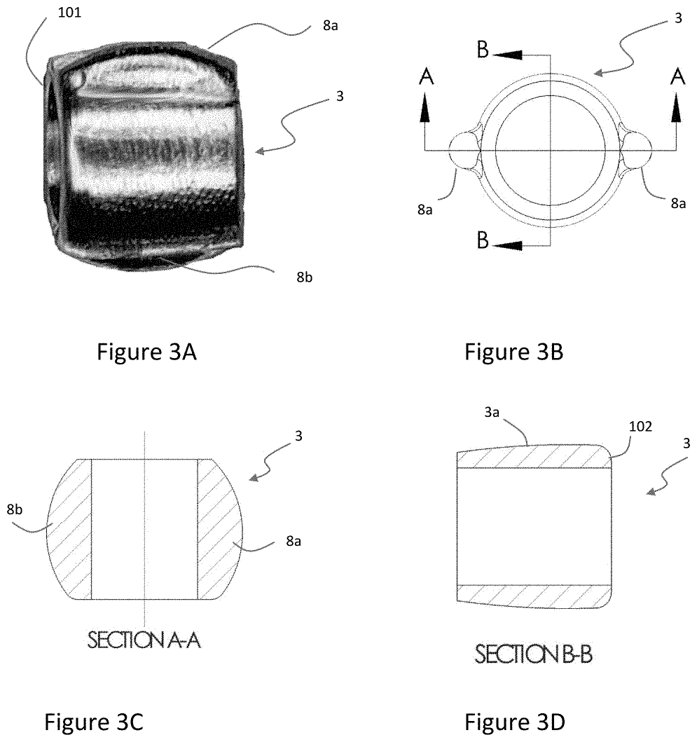

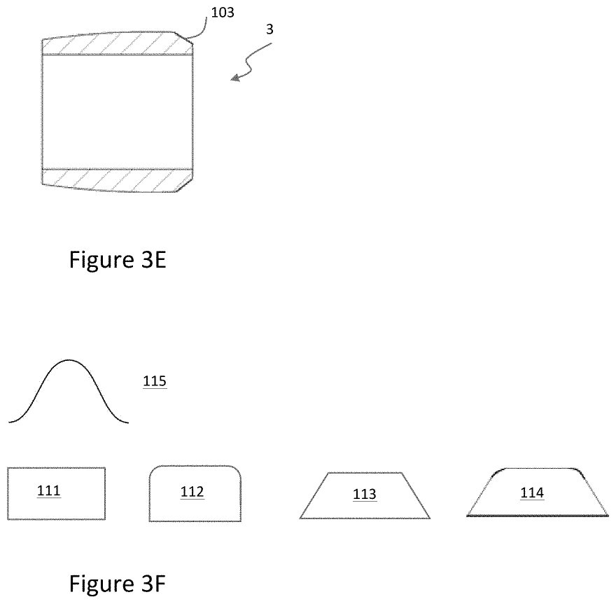

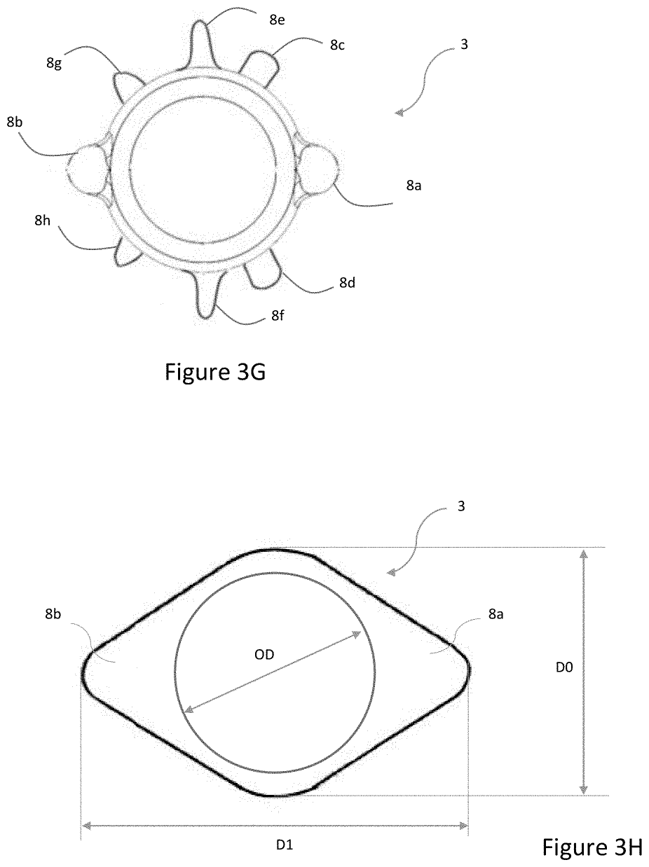

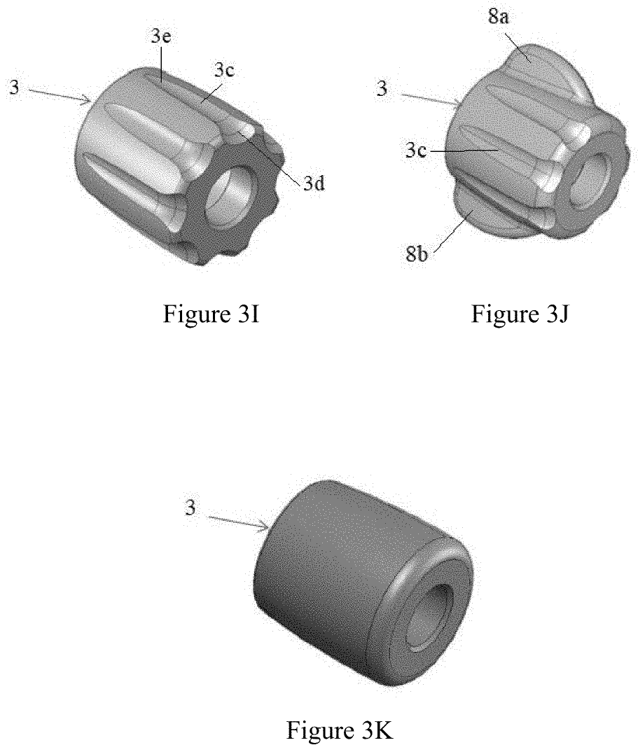

FIG. 3A shows a photomicrograph of the guide-tip 3 including two wings 8a and 8b extending from the top and bottom, respectively. The left side of the guide-tip 101 can be engaged to a distal end of a catheter as described previously. FIG. 3B shows a back view of the guide-tip. FIG. 3C is a sectional view along A-A line in FIG. 3B (through the wings 8a/8b). FIG. 3D is a cross sectional view along B-B line in FIG. 3B, showing that the base portion of tip 3 has a rounded leading edge 102. The leading edge can alternatively include a taper 103, as shown in FIG. 3E which may be smooth, i.e., have no sharp, cutting edges, enabling controlled blunt micro dissection.

The peripheral contour of the wings along the axial direction can be generally convex in shape, e.g., in the shape of a smooth elliptical curve (see FIG. 2A/2C/3A/3C). In other embodiments, and as illustrated in FIG. 3F, the side profile or shape of the wings may be rectangle (111), trapezoidal (113), or rectangular or trapezoidal with rounded outer corners (112 and 114, respectively), or sinusoidal (115).

More than two wings may be positioned around a circumference of the guide-tip 3. For example, a plurality of wings may be positioned evenly or unevenly along the circumference, and they can be arranged symmetrically or asymmetrically. The plurality of wings may be identical or different in shape and/or size. As shown in FIG. 3G, 8 wings (8a, 8b, 8c, 8d, 8e, 8f, 8g, 8h) are disposed at or near the distal end of the distal tube portion. The transverse cross sections of these wings (perpendicular to the axial direction of the distal tube portion) can vary in size and shape as shown (e.g., generally bell shape, arc, rectangle with rounded corners, etc.), where the exterior surfaces of the wings usually form a smooth transition with the outer wall of the tip.

In certain embodiments, the wings may form a continuous line with a base portion of the guide-tip. For example, as illustrated in FIG. 3H, which is a front view of a guide-tip 3, the wings 8a and 8b of tip 3 protruding laterally due to the anisotropic transverse cross-sectional shape of guide-tip 3. In such embodiment, the, the maximum transverse cross sectional width of guide-tip 3, D1, (which can be considered a “wingspan”) is greater than the minimum transverse cross-sectional width of guide-tip 3, D0. For example, D1 can be greater than D0 for about 10% to about 500%, e.g., from about 50% to about 200%. In some embodiments, D1 can be about 10%, 20%, 50%, 80%, 100%, 150%, 200%, 250%, or 300% greater than that of the inner diameter of the tip (which is about equal to the outer diameter (OD) of the distal tube portion to which the guide-tip is to be engaged).

Examples of additional guide tips 3 that may facilitate and/or improve vascular positioning, re-entry, and/or tissue delamination are shown in FIGS. 3I-3K. For example, the example of the guide tip 3 shown in FIG. 3I includes a plurality of grooves, depressions, and/or indentations 3c positioned around a circumference of the outer surface of the guide tip 3. The indentations 3c may be radially spaced around the outer surface of the guide tip 3 in substantially symmetrical or asymmetrical patterns.

The indentations 3c may have varying dimensions and/or geometric configurations to ease passage and/or delamination of subintimal layers. In the illustrated example of FIG. 3I, each indentation 3c may extend along a substantial length of the guide tip 3 in a proximal-to-distal direction. The indentations 3c may include a wider entry point or ‘mouth’ at a distal region 3d thereof and proximate to or at the distal-most end of the guide tip 3. The indentations 3c may taper into a narrower width or cross-section towards a proximal region 3e thereof proximate to or at the proximal-most end of the guide tip 3. The indentations 3c may include or define a substantially continuous arcuate surface throughout the length and width of the indentations 3c to facilitate passing through various tissue layers. The indentations 3c may be formed in the guide tip as a unitary, substantially singular solid body through molding, casting, machining, and/or other manufacturing techniques.

Turning now to FIG. 3J, an example of the guide tip 3 is shown having the indentations 3c described above combined with the wings 8a, 8b as described herein. The combined features are operable to assist in both the passage of the guide tip 3 into the tissue, as well as provide a measure of ‘roll’ orientation through both tactile feel and/or imaging modalities.

Yet another example of the guide tip 3 is illustrated in FIG. 3k. In this example, the guide tip 3 has a substantially smooth outer surface and is devoid of wings or other protrusions. The guide tip 3 may include a decreasing, tapered outer diameter in a distal-to-proximal direction to traverse one or more tissue layers in use.

The guide-tip 3 can be positioned on the distal end of the distal tube portion 11 by fusing or otherwise coupling the guide-tip 3 onto the tube wall 10. In examples where the guide-tip 3 includes one or more wings, the wings can be fabricated as an integral part of tip 3; alternatively, the wings may be fused or otherwise coupled onto a base of tip 3 by a mechanical coupling (e.g., friction), adhesion, chemical linkage, etc.

The catheter 1 may be a micro catheter having one lumen for use in conjunction with a guiding catheter. The catheter 1 may also have more than one lumen, e.g., 2, 3, 4 or 5 lumens enclosed by the tube wall 10. The lumens may have equal or unequal inner diameters. One lumen may be connected to a balloon which can be affixed to the catheter 1. A steerable guidewire may be inserted through a lumen of the catheter. The catheter may be designed to optimize parameters such as push, torque, kink performance, trackability and transition. The wall thickness of the catheter may vary along its length direction, such that the flexibility of the catheter may vary along the length direction as needed or desired.

As shown in FIGS. 2C and 2F, the tube wall 10 may be covered by a protective jacket 10a to provide a smooth outer surface while not diminishing the flexibility of the distal tube portion 11. The jacket 10a can be made from a polymer, e.g., by enclosing the tube wall 10 with a co-extruded polymeric tubular structure of single of multiple layers and heat shrinking the tubular structure, or by coating the tube wall 10 via a dip coating process. The polymer jacket material can be nylon, polyether block amide, PTFE, FEP, PFA, PET, PEEK, etc. Further, the distal tube portion 11 (or the entire length of catheter 1) may be coated with a hydrophilic polymer coating to enhance trackability. Hydrophilic polymer coatings can include polyelectrolyte and/or a non-ionic hydrophilic polymer, where the polyelectrolyte polymer can include poly(acrylamide-co-acrylic acid) salts, a poly(methacrylamide-co-acrylic acid) salts, a poly(acrylamide-co-methacrylic acid) salts, etc., and the non-ionic hydrophilic polymer may be poly(lactams), for example polyvinylpyrollidone (PVP), polyurethanes, homo- and copolymers of acrylic and methacrylic acid, polyvinyl alcohol, polyvinylethers, maleic anhydride based copolymers, polyesters, hydroxypropylcellulose, heparin, dextran, polypeptides, etc. See e.g., U.S. Pat. Nos. 6,458,867 and 8,871,869.

Also shown in FIG. 2A are two radiopaque markers 4 and 5 positioned along distal tube portion 11 for aiding radiographic visualization of the positioning of the catheter 1 in the vascular lumen. The markers can include a radiopaque material, such as metallic platinum, platinum-iridium, Ta, gold etc. in the form of wire coil or band, vapor deposition deposits, as well as radiopaque powders or fillers, e.g., barium sulfate, bismuth trioxide, bismuth sub carbonate, etc., embedded or encapsulated in a polymer matrix. Alternatively, the markers can be made from radiopaque polymers, such as radiopaque polyurethane. The markers can be in the form of bands to encircle the outer sheath of the distal tube portion 11, as shown in FIG. 2A.

As shown in FIG. 2A, the distal tube portion 11 between the marker 4 and marker 5 include a side port (or exit port) 6, which can be a through-hole on the tube wall 10. There may also be another side port 7 lying between guide-tip 3 and marker 5. (This side port can also be located proximal to marker 4). The side ports 6 and 7 can be used for exit of a re-entry wire or another re-entry device having a smaller diameter than that of the distal tube portion 11 at a direction deviating from the axis L of the distal tube portion 11. For example, the re-entry wire can have a pre-biased distal tip. As used herein, the term “pre-biased” when referring to a distal tip portion of a re-entry wire is meant that the tip portion of the re-entry wire can assume two different states, a compressed state and an uncompressed (or natural) state, where in the compressed state the distal tip portion can be axially aligned with the remainder of the wire, and in the uncompressed state the distal tip portion forms an angle (bent) with the remainder of the wire to facilitate the distal tip portion to exit from a side port of the catheter.

Side ports 6 and 7 can be positioned radially offset, between about 180° apart from each other, e.g., about 180° (±10°) apart from each other as shown in FIG. 2A. The radial displacement of the side ports relative to the wings may range from about 0° to 90°, e.g., 10, 20, 30, 50, 70 and 80 degrees. In one embodiment, the positions of the side ports may be radially offset from the wings at about 90°, as shown in FIG. 2A. In this way, when the two wings 8a/8b are positioned in a stable configuration in the subintimal space of an artery, port 6 can be facing either toward or opposing the true lumen of the artery, and the port 7 can face the opposite side.

The side ports may be symmetrical in shape and can be circular, semi-circular, ovoid, semi-ovoid, rectangular or semi-rectangular. The ports may have the same shape and size (i.e., surface area) or can be different from each other and are configured to allow for passage of a re-entry wire or another medical device through the ports. The dimensions of the port may be adjusted to accommodate different types of medical devices or wires, e.g., with diameters ranging from about 0.05 mm to about 1.0 mm. Erglis et al. Eurointervention 2010:6, 1-8. The distal tube portion 11 can contain more than two exit ports, e.g., 3, 4, 5, 6, 7, 8 . . . n ports along its length direction and radially distributed as desired.

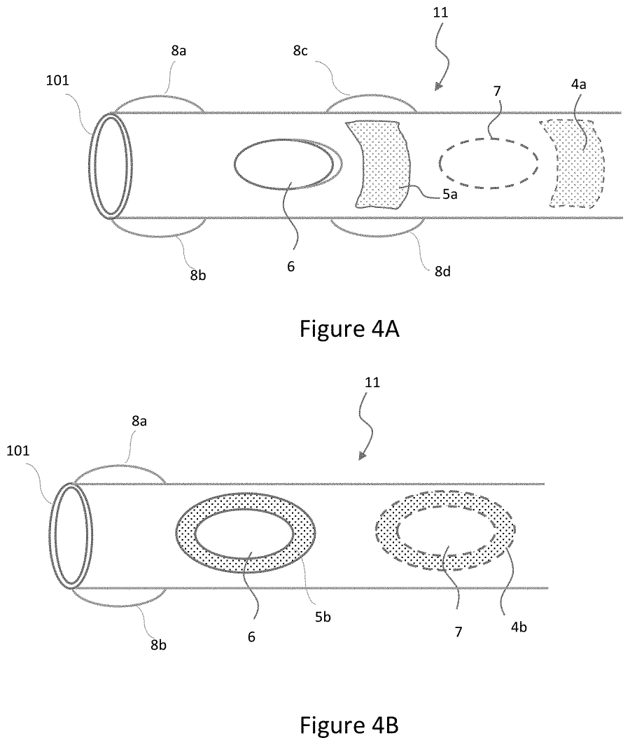

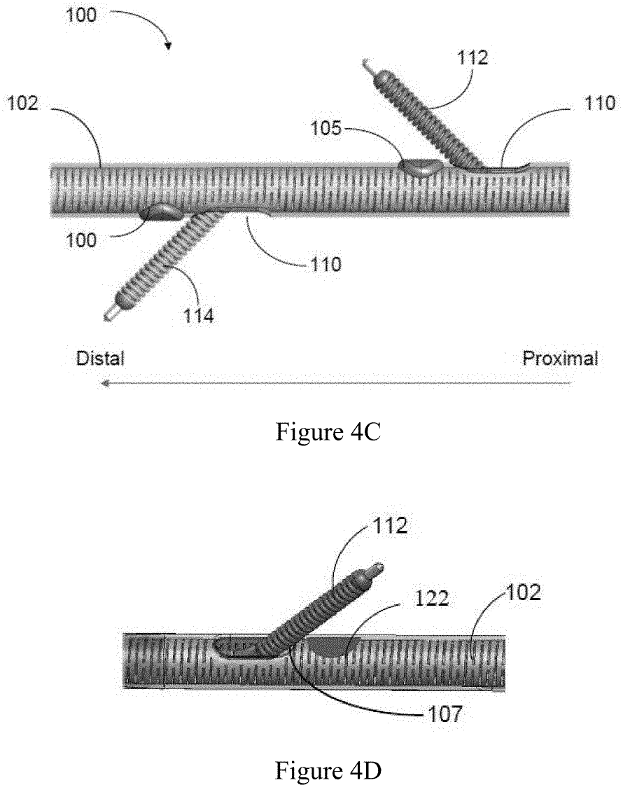

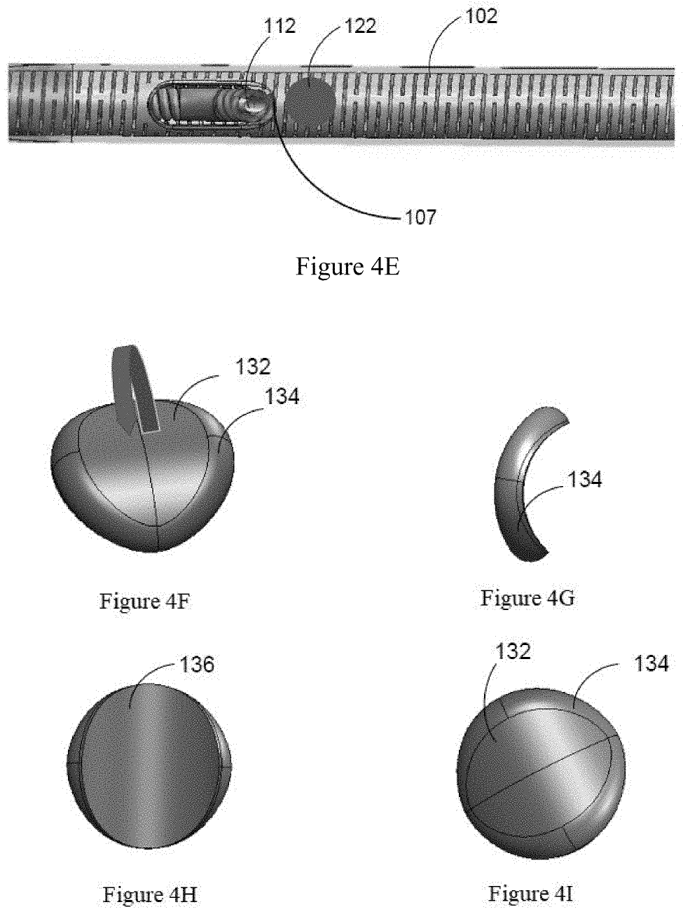

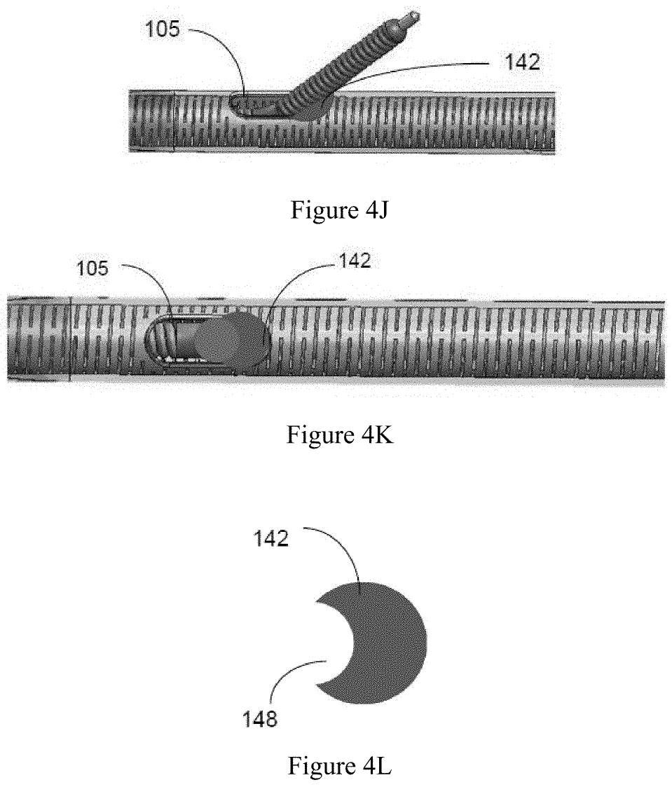

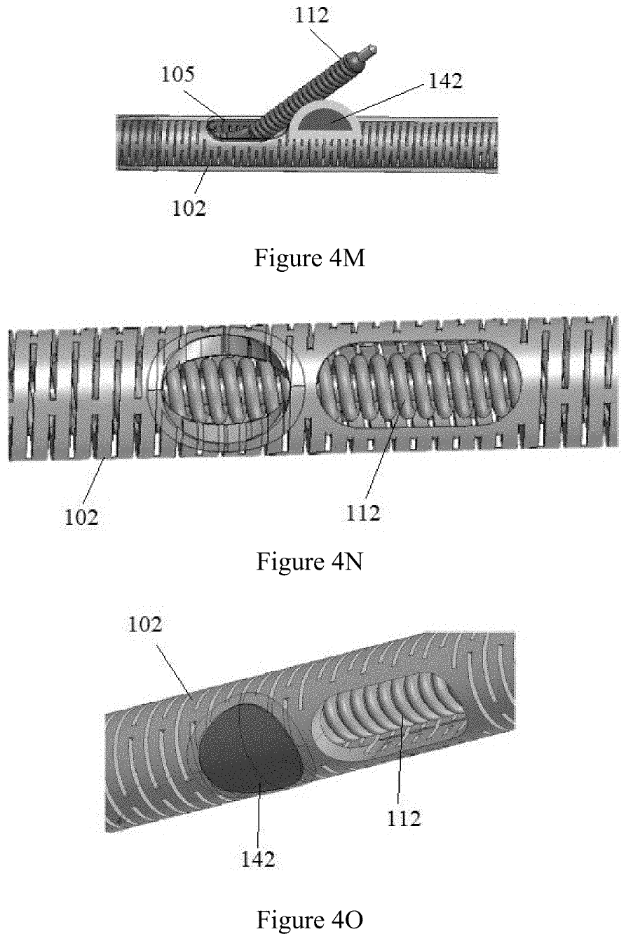

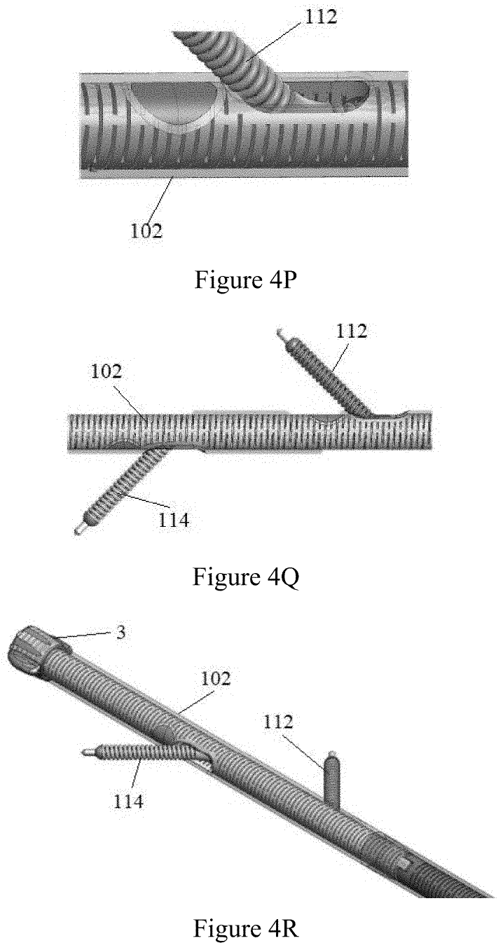

The radiopaque markers configured as bands shown in FIG. 2A can be used to facilitate determination of the positions of the side ports while the distal tube portion 11 is maneuvered in a subject's anatomy. As shown in FIG. 4A, the markers 4a and 5a (marker 5a may be positioned on the opposite side of the tube 11 and therefore is hidden from the view as shown) can also be configured as a partial band or patch which form specific alignment with a corresponding side port. For example, as shown in FIG. 4A, marker 4a is axially aligned with side port 7, whereas marker 5a is axially aligned with side port 6. Thus, like the radially opposite configuration of the side ports 6 and 7, the markers 4a and 5a are also radially opposite to each other. In this manner, visualization of the markers 4a and 5a can be used to determine the orientation of the respective side ports. The markers can be configured in different shapes, e.g., partial circumferential bands, or any other desired shapes, to facilitate determination of orientation of the ports.

As shown in FIG. 4B, the markers can be configured as surface patches 4b (which is hidden from view and shown with dashed boundary line) and 5b that enclose the circumferences of the respective exit ports 7 and 6. In such an embodiment, the marker positions that can be visualized directly correspond to the side port positions.

In either FIG. 4A or 4B, the markers should have sufficient size and suitable configuration/construction (e.g., the type of radiopaque material, load amount of radiopaque material, etc.) such that they can be visualized with the proper radiographic aid.

FIG. 4C is a side view showing a distal portion of a catheter 100 having a tube 102 having two side ports 105, 110. The tube 102 may include a tube wall having one or more features of tube wall 10 disclosed herein, and/or may include one or more polymer layers, liners, or jackets as disclosed herein. In the embodiment shown, side ports 105, 110 are positioned a distance apart longitudinally, with side port 110 distal to side port 105, and circumferentially opposite (i.e., 180° apart) to each other. A reentry device 112 (which may include, for example, a guide wire or other intravascular device) is shown extending diagonally downward through side port 105, and another reentry device 114 is shown extending diagonally upward through side port 110. In this example, a first radiopaque marker 122 is positioned longitudinally adjacent to the distal edge 107 of port 105. Radiopaque marker 122 is also circumferentially aligned with the side port 105. Similarly, a second radiopaque marker 124 is positioned longitudinally adjacent to the distal edge of side port 110. Radiopaque marker 124 is also circumferentially aligned with side port 110. Reentry devices 112, 114 may be used to pierce the intima and reenter the lumen within the vasculature. While radiopaque markers are 180° apart in the embodiment shown in FIG. 4C, in other embodiments, the markers can be positioned at other angles with respect to each other, for example, 0° to 45°, 45° to 90°, 90° to 135°, or 135° to 180°.

FIG. 4D illustrates an enlarged perspective view and FIG. 4E illustrates a top view of a side port 105, reentry device 112 and radiopaque marker 122. From a top view as shown in FIG. 4E, the marker appears in the form of a substantially circular disk, while in the perspective view of FIG. 4D, the marker follows the curvature of the tube wall and has a convex, saddle-like shape. The diameter of radiopaque markers 122, 124 can range from about 0.3 to about 0.6 mm, from about 0.4 to about 0.5 mm, or about 0.45 to about 0.46 mm. Radiopaque markers 122, 124 can be position from about 0.1 to 0.3 mm, about 0.2 to 0.4 mm about 0.3 to about 0.5 mm, or about 0.6 to 1.0 mm from the respective distal edges of the side ports to which they are adjacent 105, 110. In certain implementations the diameter of the marker 120 can be about 0.4 mm to about 0.5 mm on a catheter tube of about 0.7 to about 0.73 mm, although other sizes and proportions may be used.

FIGS. 4F, 4G, 4H, and 4I show, respectively, a front perspective view, a side perspective view, a bottom perspective view and a top perspective view of a radiopaque marker, e.g., 122, according to an embodiment of the present invention. The radiopaque marker includes a top surface 132 surrounded by an edge portion 134. As shown the top surface is slightly convex in the direction shown by the arrow in FIG. 4F. Conversely, the bottom surface 136, as viewed from the perspective in FIG. 4H, is concave. FIGS. 4F-I also show the curvilinear shape of the edge portion 134, which curves to match the contours of the cylindrical surface of the catheter tube. As noted above, the radiopaque markers can include a radiopaque material, such as metallic platinum, platinum-iridium, Ta, gold etc., as well as radiopaque powders or fillers, e.g., barium sulfate, bismuth trioxide, bismuth sub carbonate, etc., embedded or encapsulated in a polymer matrix.

FIGS. 4J and 4K show an alternative configuration and placement of a radiopaque marker 142, according to another embodiment of the present invention. In this embodiment, the radiopaque marker 142 overlaps the distal edge 107 of the side port 105. FIG. 4L shows an enlarged top view of the radiopaque maker 142, illustrating the “half-moon” shape of the marker, which is generally circular and has a semi-circular notch 148 cut out from the circumference. The diameter of radiopaque marker 142 can range from about 0.3 to about 0.7 mm, from about 0.4 to about 0.6 mm, or about 0.45 to about 0.55 mm. In some embodiments, as depicted in FIGS. 4J and 4K, the diameter of the radiopaque marker is slightly larger than the circumferential width of the side port 105, enabling the radiopaque marker 142 to be more easily seen as the catheter rotates around its longitudinal axis.

FIG. 4M illustrates another example of a placement and configuration of the marker 142. In this example, the marker 122 may be positioned distal to the side port 105/110, and may have a raised profile extending form an outer surface of the catheter 100. The marker 122 may provide a ramped or sloped surface to aid in directing the reentry device 112 out of the catheter 100 and towards a targeted tissue region.

During fabrication, the portion of the tube 102 in which the markers are positioned can be cut out of the tube using laser cutting or other manufacturing methods, as shown in FIG. 4N. The radiopaque markers, which are typically made of a different material from the catheter tube, are then inserted and fixed in the cut-out sections, an example of which is shown in FIG. 4O. Alternatively, the markers may be adhered, fused, welded, or otherwise attached directly to an outer surface of the catheter and/or components thereof (examples of which are shown in FIGS. 4C and 4M). In such examples, the markers and/or portions thereof will extend above or have a raised profile with respect to the outer surface of the catheter.

FIGS. 4P-4R provide additional illustrations of the catheter 100 in which the markers are disposed within pre-cut openings in the catheter tube, and a one or more polymer liners or outer jackets are placed over the markers and the tube wall to provide a substantially uniform, smooth exterior surface, as disclosed herein.

Also shown in FIG. 4A are additional wings 8c and 8d, which are proximal to side port 6 (wings 8a and 8b are distal to side port 6). Radiopaque material can also be included in wings 8a, 8b, and/or 8c, 8d such that these wings can also serve as radiopaque markers to help visualize the positions of the side ports. Other configurations of radiopaque markers for determining the orientation of a catheter device can also be used. See WO2010092512A1, U.S. Pat. No. 8,983,577, and U.S. Patent Application Publication No. 20140180068.

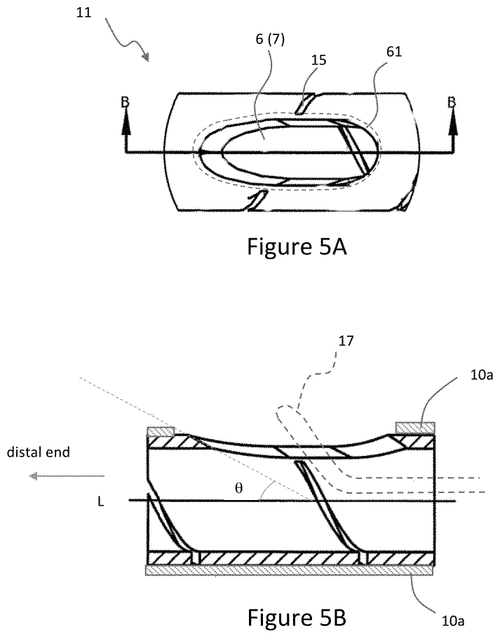

In some embodiments, the side port 6 (or 7) may be beveled, as shown in FIG. 5A (perspective view) and FIG. 5B (side cross section view along line B-B in FIG. 5A). The beveled configuration of the side port can facilitate a re-entry wire 17 with a bent tip to smoothly exit and regress from the side port (see FIG. 5B). The angle θ (see FIG. 5B) of the bevel may range from about 0° to about 90°, including, 10° to about 90°, about 20° to about 70°, or 40° to about 60°.

The configurations of the distal tube portion 11 of catheter 1 shown in FIGS. 2A-2G allow the catheter 1 to be used as an effective crossing device via subintimal exploration. The advancement of the guide-tip 3 can be affected by rotation of a proximal section of the catheter which transfers a torque to the guide-tip 3, e.g., by a torqueing device or handle coupled with an outer sheath of the catheter tubing as will be further described hereinbelow. The rotational advancement of the lateral wings 8a and 8b within the subintimal space can create a more effective delamination of layers of the blood vessel than a symmetrical blunt tip due to the presence of a controlled wide cutting or dissection plane formed by the opposing wings. Moreover, the laterally extending wings 8a/8b can facilitate orienting the catheter 1 in the subintimal space, which in conjunction with the radiopaque markers and the side ports makes it possible for the catheter 1 to also serve as an orienting device, where a pre-biased reentry wire or other type of reentry device can be manipulated and steered via the aid of radiographic visualization (e.g., x-ray fluoroscopy) to exit from one of the side ports toward the true lumen.

As shown in FIGS. 2A and 2C, the tube wall 10 of the distal tube portion 11 of catheter 1 can include a section containing a spiral cut 15 progressing about the longitudinal axis L of the tube. The spiral cut may be made using a laser, e.g., femto-second solid-state cutting laser, by removing tube material from the tube wall. A tube portion having spiral cuts can also be viewed as a ribbon or flat coil (made of portions of the remaining tube wall) wound helically about the longitudinal axis.

A spiral-cut section of the catheter can be used directly within the vasculature and may not require an outer jacket or an inner liner. Alternatively, a spiral cut section can be covered by a jacket 10a, as shown and described in connection in FIGS. 2C and 2F. Also, as shown in FIG. 5A, when located in a spiral-cut section of the distal tube portion 11, the port 6 can have a solid rim 61 that is not breached by the spiral cut 15 (in other words, the spiral cut 15 does not cut through the edge of the side port 6). As shown in FIG. 5B, if the tube wall is covered by a jacket 10a, the outer jacket 10a can be sufficiently removed around the side port so as not to interfere the re-entry wire from exiting or regressing from the side port.

The catheter may have several different spiral-cut patterns, including continuous and discontinuous. The spiral-cut sections may provide for a graduated transition in bending flexibility. For example, the spiral-cut pattern may have a pitch that changes, to increase flexibility in one or more areas. The pitch of the spiral cuts can be measured by the distance between points at the same radial position in two adjacent threads. In one embodiment, the pitch may increase as the spiral cut progresses from a proximal position to the distal end of the catheter. In another embodiment, the pitch may decrease as the spiral cut progresses from a proximal position of the catheter to the distal end of the catheter. In this case, the distal end of the catheter may be more flexible. By adjusting the pitch of the spiral cuts, the pushability, kink resistance, torque, flexibility and compression resistance of the catheter may be adjusted.

Spiral-cut sections having different cut patterns may be distributed along the length of the catheter. The spiral-cut patterns may be continuous or discontinuous along the length of the catheter. For example, there may be 1, 2, 3, 4, 5, 6, 7, . . . , n spiral-cut sections along the length of the catheter, wherein within each section a constant cut pattern may be present but across different sections the cut patterns vary, e.g., in terms of pitch. Each section may also contain a variable pitch pattern within the particular section. Each spiral-cut section may have a constant pitch, e.g., in the range of from about 0.05 mm to about 10 mm, e.g., 0.1 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 3.0 mm, 3.5 mm, 4.0 mm, etc. The pitch may also vary within each section. The pitches for different spiral-cut sections may be same or different. Alternatively, the catheter may have a continuously changing spiral-cut pattern along the length of the catheter. The orientation or handedness of spiral-cut sections in the catheter may also vary among spiral-cut sections.

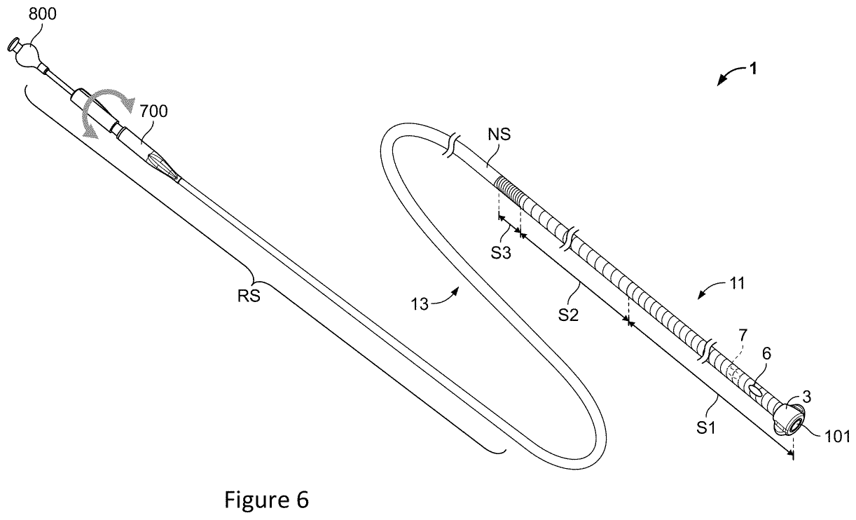

As graphically illustrated in FIG. 6 which shows a catheter 1 having a distal tube portion 11 including three consecutive spiral-cut sections, S1, S2, and S3, along its length direction. Section S1 is located at the distal end of the catheter 1, and can include a guide-tip 3 at the distal end 101 of the catheter, as well as two radially opposite and longitudinally offset side ports 6 and 7. All three spiral-cut sections can be made from a same tube (e.g., a hypotube) having a constant diameter. The distal tube portion 11 can also include an uncut portion NS proximal to spiral-cut section S3. Catheter 1 further includes a proximal tube portion 13, which can be fabricated from the same tube as distal tube portion 11, or constructed from a different tube and joined with distal tube portion 11. The proximal tube portion 13 is connected to a proximal tab 800 located at the proximal end of the catheter 1, and runs through a handle assembly or torqueing device 700, as described herein. The proximal tube portion 13 can also include a section RS which has a non-circular cross section shape (also referred to as a “railed” section, as described hereinafter) for engagement with the handle assembly 700.

Spiral sections S1, S2, S3 may each have a length and a pitch to provide a dimension and flexibility for the intended use of the catheter. For example, the lengths and pitches for each section may be selected for the performance requirements (e.g., diameter, length, shape and other configurations of the vasculature to be navigated by the catheter for accessing the treatment site) for performing a specific procedure, such as an antegrade CTO PCI procedure. For example, in one embodiment, section S1 can have a length ranging from about 10 cm-15 cm and a pitch ranging from about 0.5 mm to about 1.0 mm, section S2 can have a length ranging from about 4 to about 6 cm and a pitch ranging from about 1 to about 2 mm, section S3 can have a length ranging from about 0.5 cm to about 2 cm and a pitch ranging from 0.05 mm to about 0.3 mm.

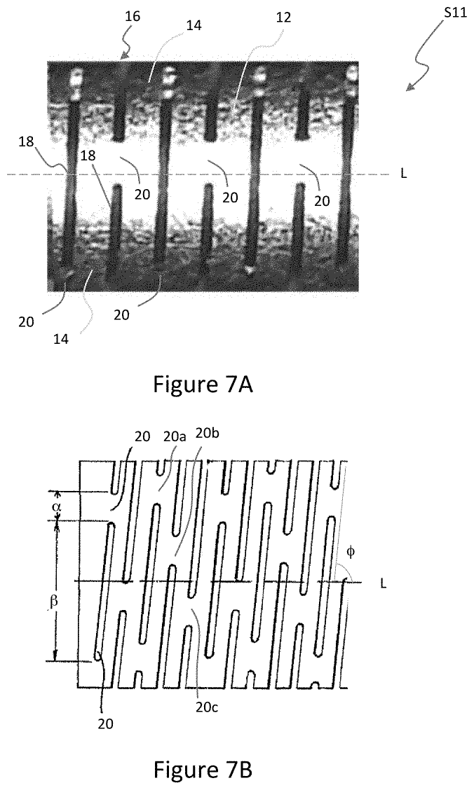

The above illustrated spiral-cuts are continuous in the spiral-cut sections. Additionally, the spiral-cuts can include a pattern of interrupted spirals, i.e., spirals that include both cut and uncut portions. As illustrated in FIGS. 7A and 7B, a spiral-cut tube section S11 of a catheter with a spiral ribbon 12 having adjacent turns 14 which are substantially defined and separated by an interrupted spiral 16, which includes alternating open or cut portions 18 and uncut portions 20. The pathway of the alternating cut and uncut sections 18 and 20 is angled with respect to a circumference of the tube portion (in other words, the pitch angle θ shown in FIG. 7B is smaller than 90 degrees). The presence of the uncut portions 20 makes the tube portion more stretch resistant than a typical wound ribbon or continuously spiral-cut tube.

Similar to what has been described with respect to continuous spiral-cuts herein, an interrupted spiral-cut pattern can also have a varying pitch that decreases from a relatively rigid region to a relatively flexible region. When a side port 6 such as one illustrated in connection with FIGS. 2A/5A is located in an interrupted spiral-cut section instead of a continuous spiral shown in FIGS. 2A/5A, the port 6 can also have a solid rim not breached by interrupted spiral cuts.

As illustrated in FIG. 7B, which depicts a portion of an unrolled catheter tube with an interrupted spiral-cut pattern, where each helically oriented uncut portion 20 has an arcuate extent “a”, and each helically oriented cut portion 18 has an arcuate extent “β”. α and β can be expressed in degrees (where each complete helical turn is 360°). The uncut portions can be distributed such that adjacent uncut portions 20 (20a, 20b, 20c) are not in axial alignment (or “staggered”) with each other along a direction parallel to the longitudinal axis L. Alternatively, uncut portions in successive helical turns can be in axial alignment to render the tube section with a bending bias. Further alternatively, as shown in FIG. 7A, the uncut portions 20 on every other turn of the interrupted spiral 16 can be axially aligned.

In some embodiments, for an interrupted spiral-cut section, the interrupted spiral pattern can be designed such that each turn or rotation of the spiral includes a specific number of cuts, Nc (e.g., 1.5, 2.5, 3.5, 4.5, 5.5, etc.). Nc can also be whole numbers, such as 2, 3, 4, 5, . . . , n, as well as other real numbers, such as 2.2, 2.4, 2.7, 3.1, 3.3, etc. At a given Nc, the uncut extent α and the cut extent β can be chosen as α=(360−(β*Nc))/Nc such that each rotation has Nc number of repeat patterns each comprising a cut portion of extent β adjacent an uncut portion of extent α. For example, at Nc=1.5, 2.5, and 3.5, the following table shows example choices of various embodiments for α and β:

| | | |
|---|---|---|
| Nc = 1.5 | Nc = 2.5 | Nc = 3.5 |

| β (°) | α (°) | β (°) | α (°) | β (°) | α (°) |
|---|---|---|---|---|---|
| 230 | 10 | 140 | 4 | 90 | 12.85714286 |
| 229 | 11 | 139 | 5 | 89 | 13.85714286 |
| 228 | 12 | 138 | 6 | 88 | 14.85714286 |
| 227 | 13 | 137 | 7 | 87 | 15.85714286 |
| 226 | 14 | 136 | 8 | 86 | 16.85714286 |
| 225 | 15 | 135 | 9 | 85 | 17.85714286 |
| 224 | 16 | 134 | 10 | 84 | 18.85714286 |
| 223 | 17 | 133 | 11 | 83 | 19.85714286 |
| 222 | 18 | 132 | 12 | 82 | 20.85714286 |
| 221 | 19 | 131 | 13 | 81 | 21.85714286 |
| 220 | 20 | 130 | 14 | 80 | 22.85714286 |
| 219 | 21 | 129 | 15 | 79 | 23.85714286 |
| 218 | 22 | 128 | 16 | 78 | 24.85714286 |
| 217 | 23 | 127 | 17 | 77 | 25.85714286 |
| 216 | 24 | 126 | 18 | 76 | 26.85714286 |
| 215 | 25 | 125 | 19 | 75 | 27.85714286 |
| 214 | 26 | 124 | 20 | 74 | 28.85714286 |
| 213 | 27 | 123 | 21 | 73 | 29.85714286 |
| 212 | 28 | 122 | 22 | 72 | 30.85714286 |
| 211 | 29 | 121 | 23 | 71 | 31.85714286 |
| 210 | 30 | 120 | 24 | 70 | 32.85714286 |
| 209 | 31 | 119 | 25 | 69 | 33.85714286 |
| 208 | 32 | 118 | 26 | 68 | 34.85714286 |
| 207 | 33 | 117 | 27 | 67 | 35.85714286 |
| 206 | 34 | 116 | 28 | 66 | 36.85714286 |
| 205 | 35 | 115 | 29 | 65 | 37.85714286 |

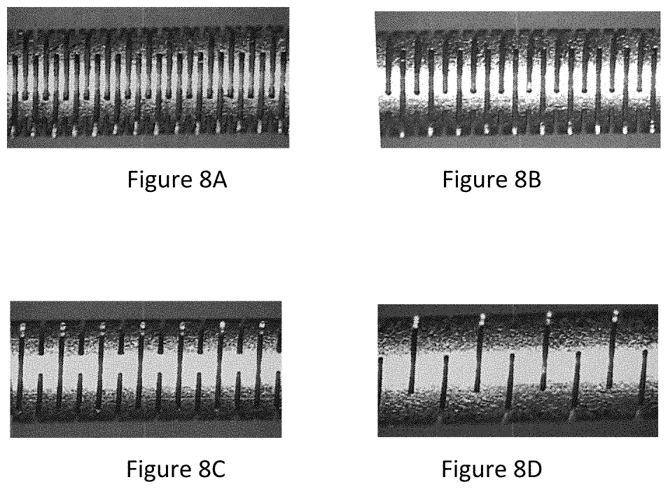

FIGS. 8A-8D are photographs of portions of a tube having interrupted spiral cuts with different pitches, as described herein.

The catheter of the present invention can include continuous spiral-cut sections (as those illustrated in FIG. 2A, 2C, 2F, 6) interrupted spiral cut sections (as those illustrated in FIGS. 7A-7C), or a hybrid of both types of spiral-cut patterns, arranged in any order.

To facilitate the transversal of a distal portion of the catheter tube 1 in the blood vessel of a subject, a torqueing device (or handle assembly) can be provided to attach to a proximal portion of the catheter tube. The handle assembly can include a lumen or internal opening to accommodate the catheter tube, as well as to frictionally engage the catheter tube to apply a torque when a portion of the handle assembly is rotated.

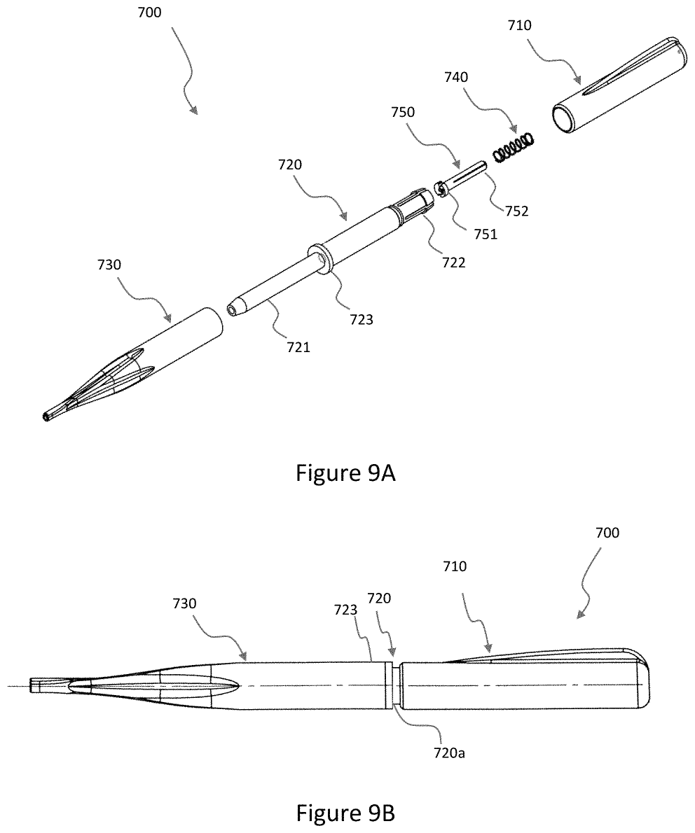

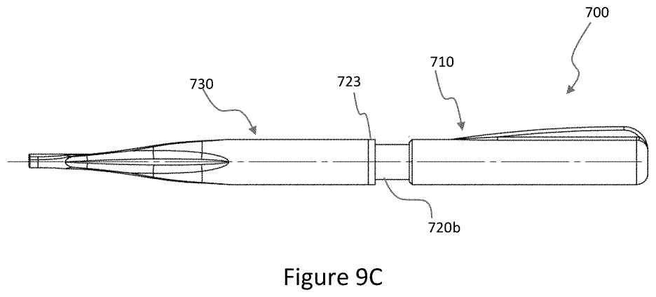

In one embodiment, and as illustrated in FIGS. 9A-9B, a handle assembly 700 includes a proximal sleeve 710, a distal outer grip 720 (which includes a distal portion 721, a proximal portion 722, and a flange 723 disposed between distal portion 721 and proximal portion 722), a distal grip sleeve 730, a spring 740, and a chuck 750 (which includes a distal flange 751 and a proximal portion 752). Each of the proximal sleeve 710, chuck 750, and distal outer grip 720 at least includes a through lumen with sufficient cross section area to allow a proximal portion 13 of catheter 1 to pass through. Additionally, the proximal sleeve 710 has a second lumen to accommodate a portion of the distal outer grip 720, and a third lumen to accommodate a portion of chuck 750. Further, the proximal portion 722 of the distal outer grip 720 includes a second lumen with a diameter to enclose the chuck 750 (including the flange 751). The spring 740 has an axial length smaller than that of the proximal portion 752 of the chuck 750, a diameter greater than that of the proximal portion 752 of the chuck 750 but smaller than the diameter of the distal flange 751 of the chuck 750.

The handle assembly 700 as assembled is shown in FIG. 9B, where the distal portion 721 of the distal outer grip 720 is covered by the distal grip sleeve 730, while the flange 723 remains visible. The proximal portion 752 of the chuck 750 is encircled by the coils of the spring 740. The chuck 750 and the spring 740 are accommodated inside the second lumen of the distal outer grip 720 as well as the third lumen of the proximal sleeve 710. The proximal sleeve 710 covers most of the proximal portion 722 of the distal outer grip 720. A short segment 720a proximal to the flange 723 of the distal outer grip 720 is exposed. This configuration shown in FIG. 9B is also referred to as a “locked” position where relative rotation of the proximal sleeve 710 and the distal grip sleeve 730 can create controlled advancement or withdrawal of the catheter within the patient's vasculature. Such rotation can be accomplished by an operator using one hand or using both hands.

Compared to known catheter systems where a torqueing handle is located at a fixed proximal position of the catheter tube, an advantage of the handle assembly of the present invention as illustrated herein is that the handle assembly can be easily unlocked or disengaged from the catheter tube so that the operator can slide the handle assembly to a different position of the catheter tube where the handle assembly can be relocked or re-engaged with the catheter tube. For example, after passing a length of the catheter tube into the vasculature of the patient, the handle assembly can be unlocked by pulling the proximal sleeve 710 away from the distal outer grip 720, resulting in an unlocked configuration where the exposed section 720b is greater than that of 720a, as shown in FIG. 9C. In this unlocked configuration, the handle assembly 700 can be slid as a whole along the railed section of the catheter to a more distal position on the catheter (i.e., further away from the proximal tab 800 and closer to the entry point of the catheter into the body of the patient), where it can be relocked by reverting back to the configuration shown in FIG. 9B. This capability of repositioning the handle assembly on different points on the catheter allows the handle assembly to be kept at a point close to the patient body, which reduces the distance between the distal tip of the catheter and the point where the torque is applied, thereby allowing for more effective transfer of torque from the point where the torque is applied to the distal tip of the catheter.

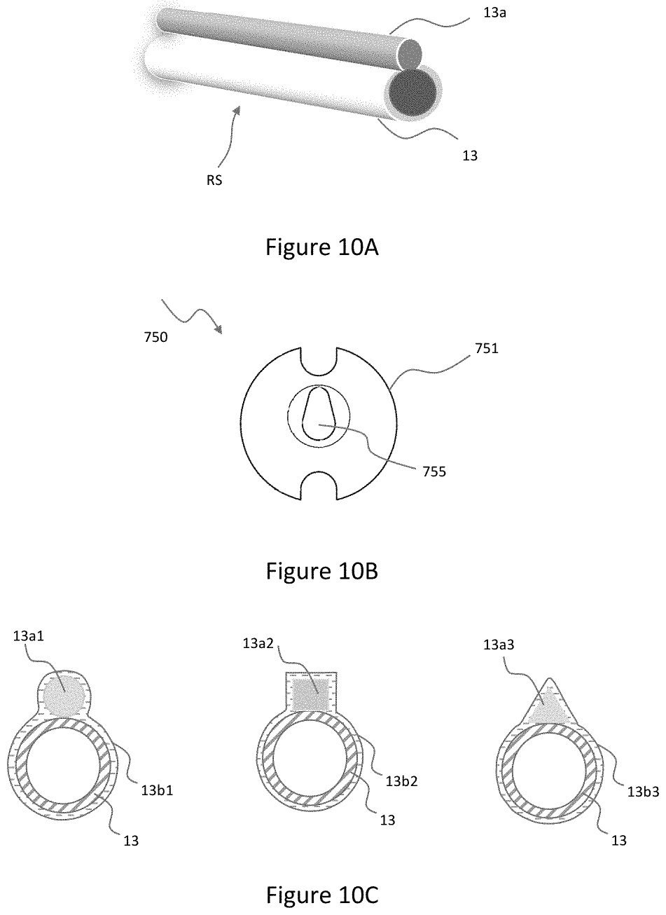

To enhance the frictional engagement between the handle assembly and the catheter and to facilitate transfer of torque from the handle assembly, a portion of the proximal tube portion 13 of the catheter can be modified to have a cross section shape that deviates from a general circular cross section shape. For example, as shown in FIG. 10A, a length of a wire or tube (either solid or hollow) 13a can be attached outside of a portion of the proximal catheter tube portion 13. The portion of the catheter tube having the attached wire or tube 13a is also referred to as a “railed” section (RS), as previously noted. The wire or tube 13a can have a size or diameter smaller than that of the proximal catheter tube portion 13, e.g., from about 5% to about 50% of the diameter of the proximal catheter tube portion 13. Alternatively, a section of the proximal portion of the catheter can be modified such that it has a non-circular cross section, in which case an externally attached wire or tube may not be needed.

As shown in FIG. 13C, the cross section of the wire or tube 13a (including 13a1, 13a2, and 13a3) can be circular (13a1) or non-circular, e.g., rectangular (13a2) or triangular (13a3), as well as other shapes, such as semi-circular, elliptical, pentagonal, or hexagonal shape, etc. The attachment between the wire or tube (13a1, 13a2, and 13a3) and the catheter tube portion 13 can be affected by providing a shrink wrapping (13b1, 13b2, 13b3) that securely encloses both the wire or tube 13a and the proximal catheter portion 13.

To accommodate the railed section of the catheter, an internal lumen of the chuck 750 and of the proximal sleeve 710 of the handle assembly can take a corresponding cross-sectional shape. For example, as illustrated in FIG. 10B, which is a front view of the chuck 750 (the front face of flange 751 is visible), where a lumen 755 for accommodating the railed section of the catheter is shown to have a shape and size that can slidably fit the overall cross section shape and size of the railed section as shown in FIG. 10A. The lumen 755 can also be shaped and sized to slidably fit any of the cross sections of the shrink wrappings 13b1, 13b2, or 13b3, as shown in FIG. 10C.

The catheter device of the present invention may be used to facilitate treatment of CTO lesions, such as in the coronary artery of a patient. First, a catheter of the present invention with a guide-tip having at least one wing (e.g., having two radially opposed wings) and a side port in a distal tube portion is advanced in the blood vessel and approaches the CTO lesion (or occlusion) in an artery. Then, the guide-tip of the catheter is advanced through the intima of the artery in a distal direction, until the at least one side port reaches a position in the subintimal space distal to the CTO lesion. In this process, the guide-tip causes dissection of the layers forming the wall of artery and establishes a channel extending longitudinally across the CTO lesion. The at least one side port can be oriented toward the true vascular lumen. Subsequently, while the guide-tip is retained in the subintimal space, a re-entry wire or device with a pre-biased distal tip portion can be introduced into the lumen of the catheter in a compressed state, and manipulated such that the distal tip of the re-entry wire or device exits from the at least one side port in a natural (uncompressed) state with the aid of radiographic visualization and enter into the true lumen.

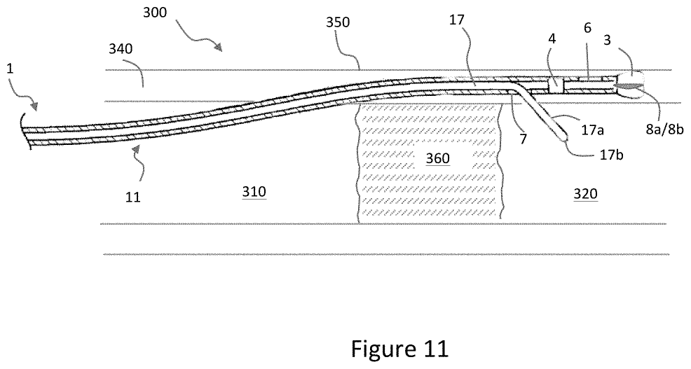

FIG. 11 depicts the final stage of this process. In the section of artery 300 having a vascular wall 350, the occlusion 360 separates the vascular lumen into a proximal segment 310 and a distal segment 320. The distal tube portion 11 of a catheter 1 has been advanced in the subintimal space 340 and a proximal side port 6 (as well as a distal side port 7) of the catheter has been advanced past the position of the occlusion 360. Radially opposed wings 8a/8b (as those shown in FIG. 2A) on the guide-tip are oriented circumferential with the vascular wall 350. The side port 6 faces toward the distal segment of vascular lumen 320. The distal tip 17b of the re-entry device 17 with a pre-biased tip portion 17a has exited from side port 7 and into the distal segment 320 of the vascular lumen with the aid of radiopaque marker 4. The distal tip 17b of the re-entry device may include a highly radiopaque material enabling it to be visualized within the catheter lumen while advancing or withdrawing the wire as well as visually enabling the operator to choose and guide the reentry wire from the correct orientation out of the side ports under fluoroscopic guidance.

In the above approach where the re-entry device or wire having a pre-biased tip is introduced into the true lumen via a side port, one or more side ports may be utilized during the reentry manipulation. For example, for a catheter having two radially opposed side ports and two corresponding radiopaque markers as illustrated in FIG. 2A, the reentry wire may be introduced into the true lumen by a first attempt to penetrate the pre-biased tip of the re-entry wire through either side port. If the first attempt is not successful, the re-entry wire is withdrawn from that side port and a second attempt can be made to manipulate the tip of the reentry device to exit from the other side port while maintaining the position and orientation of the wings of the catheter. The second attempt is expected to be successful because the orientation of the exit ports is such that one exit port faces toward the true lumen and the other faces the opposite side. Such reentry can also be accomplished using only one side port, where if the first attempt is unsuccessful, the catheter can be rotated for about 180 degrees within the subintimal space to arrive at another stable position, and the reentry is attempted again, which is expected to be successful. The radiopaque markers illustrated in connection with FIGS. 4A and 4B can also be used to determine the orientation of the catheter and the side ports for manipulation of the reentry wire to enter into the true lumen.

The scope of the present invention is not limited by what has been specifically shown and described hereinabove. Those skilled in the art will recognize that there are suitable alternatives to the depicted examples of configurations, constructions, and dimensions, and materials. The citation and discussion of any references in the application is provided merely to clarify the description of the present invention and is not an admission that any reference is prior art to the invention described herein. All references cited and discussed in this specification are incorporated herein by reference in their entirety. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

It will be appreciated by persons skilled in the art that the present disclosure is not limited to what has been particularly shown and described herein above. In addition, unless mention was made above to the contrary, it should be noted that all of the accompanying drawings are not to scale. Of note, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein. Moreover, while certain embodiments or figures described herein may illustrate features not expressly indicated on other figures or embodiments, it is understood that the features and components of the examples disclosed herein are not necessarily exclusive of each other and may be included in a variety of different combinations or configurations without departing from the scope and spirit of the disclosure. A variety of modifications and variations are possible in light of the above teachings without departing from the scope and spirit of the disclosure, which is limited only by the following claims.

What is claimed is:

1. A catheter device comprising:
a distal tube portion having a longitudinal axis and a tube wall comprising at least one side port; and
a guide tip mounted on the distal tube portion, the guide tip defining:
a unitary body having an outer wall, and
a plurality of indentations in the outer wall, wherein the indentations are oriented substantially parallel to the longitudinal axis, and wherein each indentation extends from a distal-most end of the guide tip to a proximal region of the guide tip.

2. The catheter device of claim 1, wherein each of the plurality of indentations extends along a substantial length of the guide tip in a proximal-to-distal direction.

3. The catheter device of claim 1, wherein each of the plurality of indentations defines an axial length smaller than an overall axial length of the guide tip.

4. The catheter device of claim 1, wherein each of the plurality of indentations defines a width that varies along an axial length thereof.

5. The catheter device of claim 4, wherein each of the plurality of indentations defines a width at a distal region thereof that is greater than a width at a proximal region thereof.

6. The catheter device of claim 1, wherein each of the plurality of indentations defines a substantially arcuate surface.

7. The catheter device of claim 1, wherein each of the plurality of indentations defines a height with respect to the outer wall that varies along an axial length of the indentation.

8. The catheter device of claim 1, wherein the plurality of indentations includes 4 or more equally-spaced indentations.

9. The catheter device of claim 1, wherein each of the plurality of indentations has a uniform shape.

10. The catheter device of claim 1, wherein the plurality of indentations is evenly spaced around a circumference of the guide tip.

11. The catheter device of claim 1, comprising a first side port and a second side port, wherein the second side port is located distal to the first side port and being longitudinally and radially offset from the first side port.

12. The catheter device of claim 11, further comprising a first radiopaque marker located longitudinally between the first side port and the second side port, and a second radiopaque marker positioned distal to the second side port.

13. The catheter device of claim 12, wherein the first side port and the second side port are radially offset for about 180° degrees from each other.

14. The catheter device of claim 1, further comprising a radiopaque marker affixed on the distal tube portion in axial alignment with the at least one side port.

15. The catheter device of claim 1, further comprising a radiopaque marker encircling the at least one side port.

16. The catheter device of claim 1, wherein the at least one side port is beveled.

17. The catheter device of claim 1, wherein the guide tip is formed from a metal.

18. A catheter device comprising:
a distal tube portion having a longitudinal axis and a tube wall comprising at least one side port; and
a guide tip coaxially mounted on the distal end of the distal tube portion, the guide tip defining:
a base portion defining a circumference and a distal-most face transverse to the longitudinal axis, and
a plurality of indentations equally spaced around the circumference of the base portion, wherein each of the indentations defines a width at a distal region thereof that is greater than a width at a proximal region thereof, and wherein each of the indentations extends from the distal-most face of the base portion.
